(19) United States
(12) Patent Application Publication
(10) Pub. No.: US 2020/0188350 A1
(43) Pub. Date: Jun. 18, 2020

(54) BIOACTIVE MOLECULES FROM OROXYLUM INDICUM AND THEIR THERAPEUTIC APPLICATIONS

(71) Applicants: Muhammed Majeed, Bangalore (IN); Kalyanam Nagabhushanam, East Windsor, NJ (US)

(72) Inventors: Muhammed Majeed, Bangalore (IN); Kalyanam Nagabhushanam, East Windsor, NJ (US)

(21) Appl. No.: 16/801,257

(22) Filed: Feb. 26, 2020

Related U.S. Application Data
(63) Continuation-in-part of application No. 16/441,465, filed on Jun. 14, 2019.
(60) Provisional application No. 62/685,400, filed on Jun. 15, 2018.

(51) Int. Cl. A61K 31/353; A61K 9/00

(52) U.S. Cl. CPC A61K 31/353; A61K 9/0053

(57) ABSTRACT

The present invention discloses a composition comprising not less than 10% w/w of oroxylin A, not less than 10% w/w of baicalein and not less than 2% w/w of chrysin for use in the therapeutic management of epilepsy.

### CROSS-REFERENCE TO RELATED PATENT APPLICATIONS

This application is a continuation-in-part of U.S. patent application Ser. No. 16/441,465 filed on 14 Jun. 2019 which in turn is a non-provisional filing of U.S. provisional patent application No. 62/685,400 filed on 15 Jun. 2018, which is being incorporated herein in its entirety.

### BACKGROUND OF THE INVENTION

### Field of the Invention

The invention in general relates to compositions for therapeutic management of epilepsy and hypertriglyceridemia. More specifically the present invention relates to therapeutic management of epilepsy and hypertriglyceridemia using a composition comprising oroxylin A, baicalein and chrysin (OBC).

### Description of Prior Art

Triglycerides are a class of lipid molecules which are stored in the fat cells and released at the time of energy requirement. The body converts die calories into triglycerides and stores in the adipocytes. Hypertriglyceridemia is a clinical condition wherein there is an increase in circulating levels of triglycerides. If the calorie intake exceeds the output, there will be an increase in the levels of triglycerides in blood.

Evidence indicates that elevated triglyceride levels contribute to the increased risk of cardiovascular disease and stroke. Hypertriglyceridemia is associated with an increased risk of acute pancreatitis. The following prior art documents discuss about the causes and effects of hypertriglyceridemia
a) Yuan ct al., Hypertriglyceridemia: its etiology, effects and treatment, CMAJ. 2007; 176(8): 1113-1120.
b) Robertson S, Hypertriglyceridemia Cause and Symptoms, https.//www.news-medical.net/health/Hypertriglyceridemia-Cause-and-Symptoms.aspx, accessed 10 Jun. 2019
c) John D. Brunzell. Hypertriglyceridemia, N Engl J Med 2007; 357:1009-1017
d) Ceriello et al., Evidence for an Independent and Cumulative Effect of Postprandial Hypertriglyceridemia and Hyperglycemia on Endothelial Dysfunction and Oxidative Stress Generation, Circulation. 2002; 106:1211-1218

Hypertriglyceridemia is often co-moibid with and/or indicate other clinical conditions like diabetes, hyperglycemia, hypothyroidism, metabolic syndrome, obesity, and genetic conditions. It is also present as a side effect of taking certain medications like, chemotherapeutics, diuretics, steroids, beta blockers etc, which can be easily reversed (Triglycerides: Why do they matter?, Mayo Clinic, https://www.mayoclinic.org/diseases-conditions/high-blood-cholesterol/in-depth/triglycerides/art-20048186, accessed 10 Jun. 2019). There are many treatment strategies employed for the management and treatment of hypertriglyceridemia. Natural molecules that can decrease the levels of triglycerides in blood are now being increasingly evaluated. Some of the natural molecules that are reported to reduce triglyceride levels are listed below:
1. Rideout et al., Triglyceride-Lowering Response To Plant Sterol and Stanol Consumption, J AOAC Int. 2015; 98(3): 707-715.
2. Schonewille et al., Serum TG-lowering properties of plant sterols and stanols are associated with decreased hepatic VLDL secretion. J Lipid Res. 2014; 55(12): 2554-2561.
3. Vallianou et al., Camphene, a Plant-Derived Monoterpene. Reduces Plasma Cholesterol and Triglycerides in Hyperlipidemic Rats Independently of HMG-CoA Reductase Activity, PLoS ONE 6(11): e20516.
4. Majeed et al., Composition comprising scirpusin A and scirpusin B and anti-obesity potential thereof, U.S. patent Ser. No. 10/172,903.
5. Majeed et al., Method for the treatment of hypercholesterolemia. U.S. Pat. No. 9,610,273.

Epilepsy is a brain disorder characterized by the presence of epileptic seizures, generated by the neurobiologic, cognitive, psychological, and social consequences (Fisher et el., Epileptic seizures and epilepsy: definitions proposed by the International League Against Epilepsy (ILAE) and the International Bureau for Epilepsy (IBE). Epilepsia. 2005 April; 46(4): 470-2). It is a condition with a transient occurrence of signs and/or symptoms due to abnormal excessive or synchronous neuronal activity in the brain. Most epileptic seizures are characterised by reduced locomotion, increased muscle rigidity, salivation, teeth grinding, clonus and repetitive head/leg movement. There are many types of seizures namely absence seizures (formerly known as petit mal), tonic-clonic or convulsive seizures (formerly known as grand mal), atonic seizures (also known as drop attacks), clonic seizures, tonic seizures and myoclonic seizures.

The causes for the development of seizure are manifold. It varies with age and other pathological conditions. The epilepsy foundation of USA discloses the different causes of development of epileptic seizures (https://www.epilepsy.com/learn/about-epilepsy-basics/what-causes-epilepsy-and-seizures) in newborn, children, adults and in seniors with stroke, trauma and neurodegenerative disorders like Alzheimer's disease. The common causes of seizures in newborns include brain malformations, lack of oxygen during birth, low levels of blood sugar, blood calcium, blood magnesium or other electrolyte problem, inborn errors of metabolism, intracranial hemorrhage and maternal drug use. In children and adults it occurs due to fever (febrile seizures), brain tumor (rarely), infections, congenital conditions (down's syndrome; angelman's syndrome; tuberous sclerosis and neurofibromatosis), genetic and biochemical factors and deficiencies, progressive brain disease (rare) and head trauma.

The treatment modalities for the management of epileptic seizures include administration of drugs according to the type of seizure, age, sex and other medical conditions. However, most of the anti-epileptic drugs cause many side effects which include tiredness, dizziness, weight gain, thinning bones, rashes, clumsiness, trouble talking, trouble remembering things, trouble thinking, mood changes, and weight loss. A natural and safe alternative is warranted to ameliorate the symptoms of epilepsy. Plant derived molecules for the management of epilepsy have been already reported. However there is a lack of evidence for efficacy and toxicity for most of the molecules. Plants and plant derived molecules obtained from Lavandula officinalis, Zizyphus jujube, Taxus wallichiana Zucc, Ganoderma lucidum, Uncaria rhynchophylla have been reported to be effective in the management of epilepsy. (Wei Liu et al., The effects of herbal medicine on epilepsy, Oncotarget. 2017 Jul. 18; 8(29): 48385-48397). Bioactive flavones isolated from Scutellaria baicalensis have been tested for convulsion related activities. Baicalin exhibited anticonvulsant effects in electrogenic response score system and the pentylenetetrazole seizure model. (Yoon et al., Convulsion-related activities of Scutellaria flavones are related to the 5,7-dihydroxyl structures, Eur J Pharmacol. 2011 Jun. 1; 659(2-3):155-60).

However, a natural molecule and/or a combination of natural molecules that is effective in reducing epileptic seizures and decrease triglycerides, especially in hypertriglyceridemia induced by chemotherapeutics and hyperglycemia are lacking. The present invention solves the above problem by disclosing a composition comprising oroxyiin A, baicalein and chrysin for management of epilepsy and hypertriglyceridemia.

It is a principle objective of the invention to disclose a method for therapeutic management of epilepsy using a composition comprising oroxyiin A, baicalein and chrysin.

It is another objective of the invention to disclose a method for therapeutic management of hypertriglyceridemia using a composition comprising oroxyiin A, baicalein and chrysin.

The invention fulfils the above mentioned objective and provides further related advantages.

### SUMMARY OF THE INVENTION

The present invention discloses a composition comprising not less than 10% w/w of oroxyiin A, not less than 10% w/w of baicalein and not less than 2% w/w of chrysin for use in the therapeutic management of epileptic seizures. The invention also discloses the use of a composition comprising not less than 10% w/w of oroxyiin A, not less than 10% w/w of baicalein and not less than 2% w/w of chrysin for use in the therapeutic management hypertriglyceridemia associated with chemotherapy and hyperglycemia.

### BRIEF DESCRIPTION OF THE FIGURE

FIG. 1 is graphical representation of effect of composition comprising oroxyiin A, baicalein and chrysin (OBC) (250 and 500 mg/kg, oral administration, 2 weeks) on the kainic acid (KA)-induced seizures as seen by the decrease in Racine score with increase in concentration of the composition.

### DESCRIPTION OF THE MOST PREFERRED EMBODIMENTS

In the most preferred embodiment the invention discloses a method of therapeutic management of hypertriglyceridemia in mammals, said method comprising steps of administering effective concentration of a composition comprising not less than 10% w/w of oroxylin A, not less than 10% w/w of baicalein and not less than 2% w/w of chrysin, to mammals in need of such therapeutic management. In a related embodiment, the composition preferably comprises 10%-15% w/w of oroxylin A, 10%-25% w/w of baicalein and 2%-10% w/w of chrysin. In a related embodiment, hypertriglyceridemia is caused by chemotherapeutics and hyperglycemia. In a preferred embodiment, the mammal is human. In another preferred embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a composition comprising not less than 10% w/w of oroxylin A, not less than 10% w/w of baicalein and not less than 2% w/w of chrysin, for use in the therapeutic management of hypertriglyceridemia in mammals. In a related embodiment, the composition preferably comprises 10%-15% w/w of oroxylin A, 10%-25% w/w of baicalein and 2%-10% w/w of chrysin. In a related embodiment, hypertriglyceridemia is caused by chemotherapeutics and hyperglycemia. In another preferred embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables. In another related embodiment, the mammal is human.

In the most preferred embodiment the invention discloses a method of therapeutic management of epileptic seizures in mammals, said method comprising steps of administering effective concentration of a composition comprising not less than 10% w/w of oroxylin A, not less than 10% w/w of baicalein and not less than 2% w/w of chrysin, to mammals in need of such therapeutic management to mammals in need of such therapeutic management to bring about a reduction in the severity and occurrence of seizures. In a related embodiment, the composition preferably comprises 0%-15% w/w of oroxylin A. 10%-25% w/w of baicalein and 2%-10% w/w of chrysin. In a preferred embodiment, the mammal is human. In another preferred embodiment, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.

In another preferred embodiment, the invention discloses a composition comprising not less than 10% w/w of oroxylin A, not less than 10% w/w of baicalein and not less than 2% w/w of chrysin, for use in the therapeutic management of epileptic seizures in mammals. In a related aspect, the composition preferably comprises 10%-15% w/w of oroxylin A, 10%-25% w/w of baicalein and 2%-10% w/w of chrysin. In another related aspect, the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables. In yet another related aspect, the mammal is human.

The specific examples included herein below illustrate the aforesaid most preferred embodiments of the present invention.

### Example 1: Hypotriglyceridemic Effects of Oroxylin a, Baicalein and Chrysin (OBC) on Chemotherapy Induced Memory Impairment

The composition comprising oroxylin A, baicalein and chrysin (OBC), was isolated from Oroxylum indicum as per the process mentioned in U.S. patent application Ser. No. 15/805,320.

Methodology: Mice received intraperitoneal (IP) injection of saline or chemotherapeutics (CT)—doxorubicin-2 mg/kg & cyclophosphamide-50 mg/kg one injection/week for 4 weeks. The composition comprising oroxylin A, baicalein and chrysin [250 mg/kg—low dose (LD) and 500 mg/kg—high dose (HD)] was mixed with powdered rodent food and fed daily for 4 weeks.

The mice serum samples were tested using an automated machine for the determination of hematological markers and other parameters to monitor liver and kidney functions.

TABLE 1
Hematological markers

| | | | CT + OBC | CT + OBC |
|---|---|---|---|---|
| Parameters | Control | CT | (LD) | (HD) |
| Glucose | 165.00 ± 6.59 | 157.33 ± 3.59 | 202.33 ± 1.03 | 178 ± 0.44 |
| Cholesterol | 126.00 ± 1.79 | 126.66 ± 2.46 | 118 ± 1.18 | 115.33 ± 0.68 |
| Triglyceride | 166.33 ± 1.37 | 179.67 ± 2.29* | 142.67 ± 2.62 | 137.67 ± 1.81 |

*Indicates significant change p < 0.05

Chemotherapeutics significantly elevated triglyceride levels and OBC prevented chemotherapeutics effect. OBC may protect from drug-induced elevation of triglyceride levels.

### Example 2: Effects of Composition Comprising Oroxylin A, Baicalein and Chrysin (OBC) on Hyperglycemia Induced Triglyceride Elevation

The composition comprising oroxylin A, baicalein and chrysin (OBC), was isolated from Oroxylum indicum as per the process mentioned in U.S. patent application Ser. No. 15/805,320.

Methodology: Rats received intraperitoneal (IP) injection of saline or streptozotocin (STZ) (55 mg/kg). The composition comprising oroxylin A, baicalein and chrysin (OBC) [250 mg/kg—low dose (LD) and 500 mg/kg—high dose (HD)] was mixed with powdered rodent food and fed daily for 4 weeks.

Blood samples were withdrawn from the rats before euthanization. These samples were immediately tested using an automated machine for the determination of hematological markers and other parameters to monitor liver and kidney functions.

TABLE 2
Hematological Markers

| | | | STZ + OBC | STZ + OBC |
|---|---|---|---|---|
| Parameters | Control | STZ | (LD) | (HD) |
| Glucose | 190.4 ± 4.56 | 602.4 ± 48.79* | 509 ± 57.53 | 561.8 ± |
| | | | | 29.91 |
| Cholesterol | 121.8 ± 3.07 | 141.6 ± 10.48 | 130.6 ± 8.22 | 151 ± |
| | | | | 6.98 |
| Triglyceride | 129.8 ± 5.43 | 633.2 ± 156.9* | 334.2 ± 88.11 | 551.2 ± |
| | | | | 65.05 |

*represent significant change p < 0.05

OBC significantly decreases the STZ-increased triglyceride levels. These results suggest that OBC may be useful in the management of blood triglyceride levels.

In conclusion, the composition comprising oroxylin A, baicalein and chrysin was very effective in ameliorating the symptoms of chemotherapy and hyperglycemia induced triglyceride elevation. U.S. patent application Ser. No. 16/009,490 and PCT application no. PCT/US18/37724 discloses the use of comprising oroxylin A, baicalein and chrysin in ameliorating the symptoms of chemotherapy and hyperglycemia induced cognitive dysfunction and memory impairment. A large body of evidence indicate that the enzyme monoamine oxidase (MAO) play an important role in neuroprotection. However, the exact mechanisms underlying the protective effects of MAOs in the brain are still unknown. Although reports indicate that over expression of MAOs in neurodegenerative diseases like Alzheimer's disease. Parkinson's disease and epileptic seizures, deficiency of the enzyme is also reported in the above mentioned conditions. The treatment methods that are generally employed are aimed at inhibiting the activity of MAOs and bioactive molecules that increase the activity of MAOs for ameliorating the symptoms are lacking. The composition comprising oroxylin A, baicalein and chrysin may also be very effective in modulating the levels of MAOs, which can be used for the management of many neurological diseases, specifically epilepsy.

### Example 3: Anti-Epileptic Activity

Two-month-old male Sprague-Dawley rats were purchased from commercial vendor and housed at 25°±2° C., with the humidity maintained at 55%, wood chip bedding, on a 12:12 hour dark/light cycle, with free access to food and water. All animal procedures were carried out in accordance with NIH and Auburn University Animal Care and Use Committee guidelines. The rats were divided into four groups (1. Control, 2. Kainic acid (10 mg/kg), 3. LD (Low Dose)-composition containing OBC 250 mg/kg+Kainic acid, and 4. High Dose (HD)—composition containing OBC 500 mg/kg+Kainic acid). Composition containing OBC was mixed with powdered feed and administered orally for 2 weeks prior to kainic-acid administration. Administration of kainic acid has been shown to increase seizures as seen by increased Racine scores (Dawson and Wallace, Kainic acid-induced seizures in aged rats: neurochemical correlates. Brain Res Bull. 1992; 29(3-4):459-68; Eppler et al., Kainic acid (KA)-induced seizures in Sprague-Dawley rats and the effect of dietary taurine (TAU) supplementation or deficiency. Amino Acids. 1999; 16(2): 133-47; Sharma et al., Mesial temporal lobe epilepsy: pathogenesis, induced rodent models and lesions. Toxicol Pathol. 2007; 35(7):984-99; French et al., Intrahippocampal kainic acid, seizures and local neuronal degeneration: relationships assessed in unanesthetized rats. Neuroscience. 1982; 7(10):2525-36; Jinde et al., Lack of kainic acid-induced gamma oscillations predicts subsequent CA1 excitotoxic cell death. Eur J Neurosci. 2009; 30(6): 1036-55; Medvedev et al., Kainic acid induces distinct types of epileptiform discharge with differential involvement of hippocampus and neocortex. Brain Res Bull. 2000; 52(2):89-98). Typically, Racine scores include the following stages:
1. Mouth and facial movement
2. Head nodding
3. Forelimb clonus
4. Rearing with forelimb clonus
5. Rearing and falling with forelimb clonus (generalized motor convulsions)
   Racine scores are acceptable scoring scale to validate the neuroprotective effects of synthetic or natural drugs for their efficacy against seizures.

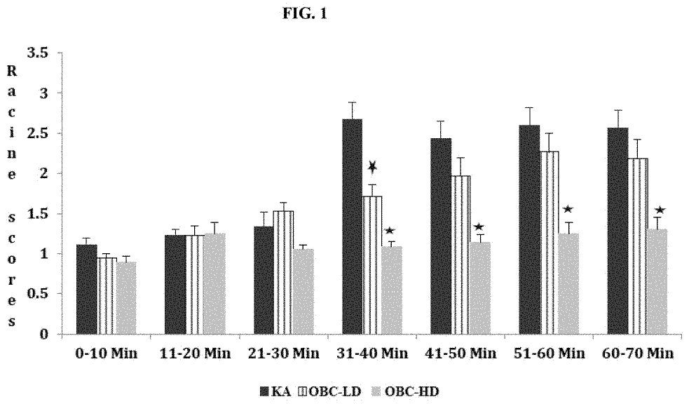

In the present study, composition containing OBC (250 and 500 mg/kg, oral administration) dose dependency reduced the plethora of seizure activities induced by kainic acid (FIG. 1). Composition containing OBC significantly improved the reduced locomotion, increased muscle rigidity, salivation, teeth grinding, clonus and repetitive head/leg movement which are evaluated as a part of Racine scores.

Seizures have shown to affect the psychological and social behaviors, movement and consciousness. Untreated seizures have shown to increase the risk for morbidity and mortality. Composition containing OBC exhibits anti-seizure activity in addition to their neuroprotective effects (antioxidant, mitochondrial enhancing properties). Composition containing OBC can be used for the management of various types of seizures such as absence seizures (formerly known as petit mal), tonic-clonic or convulsive seizures (formerly known as grand mal), atonic seizures (also known as drop attacks), clonic seizures, tonic seizures and myoclonic seizures.

While the invention has been described with reference to a preferred embodiment, it is to be clearly understood by those skilled in the art that the invention is not limited thereto. Rather, die scope of the invention is to be interpreted only in conjunction with the appended claims.

We claim:

1. A method of therapeutic management of epileptic seizures in mammals, said method comprising steps of administering elective concentration of a composition comprising not less than 10% w/w of oroxylin A, not less than 10% w/w of baicalein and not less than 2% w/w of chrysin to mammals in need of such therapeutic management, to bring about a reduction in the severity and occurrence of seizures.

2. The method as in claim 1, wherein the composition comprises 10%-15% w/w of oroxylin A, 10%-25% w/w of baicalein and 2%-10% w/w of chrysin.

3. The method as in claim 1, wherein the mammal is human.

4. The method as in claim 1, wherein the composition is formulated with pharmaceutically/nutraceutically acceptable excipients, adjuvants, diluents or carriers and administered orally in the form of tablets, capsules, syrups, gummies, powders, suspensions, emulsions, chewables, candies and eatables.
